(19) United States
(12) Patent Application Publication
(10) Pub. No.: US 2008/0300397 A1
(43) Pub. Date: Dec. 4, 2008

(54) MODIFIED SPIN COLUMN FOR SIMPLE AND RAPID PLASMID DNA EXTRACTION

(75) Inventors: Michael Kenneth KENRICK, Caerphilly (GB); Christopher JONES, Monmount (GB); Malcolm John HATCHER, Caerphilly (GB); Alison HOPKINS, Newport (GB); Peter James TATNELL, Caerphilly (GB); David WILLIAMS, Barry (GB)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE UK LIMITED, BUCKINGHAMSHIRE (GB)

(21) Appl. No.: 12/128,706

(22) Filed: May 29, 2008

Related U.S. Application Data
(60) Provisional application No. 60/941,032, filed on May 31, 2007.

(51) Int. Cl. C07H 1/06; B01D 29/50

(52) U.S. Cl. 536/25.42; 210/203

(57) ABSTRACT

The invention relates to a modified spin column for the isolation and purification of plasmid DNA. A pre-filtration disc is included in a traditional spin column. During plasmid DNA isolation, the lysate can be loaded directly to the modified spin column, eliminates the need to first remove the flocculants containing cellular debris. This results in a much shortened process. Variation of the invention includes a depth filter in between the pre-filtration disc and the main separation matrix. Also provided are kits for isolation of plasmid DNA including the modified spin columns.

### CROSS-REFERENCE TO RELATED APPLICATIONS

This application claims priority to U.S. provisional patent application No. 60/941,032 filed 31 May 2007; the disclosure of which is incorporated herein by reference in it entirety.

### FIELD OF THE INVENTION

This invention relates to an improved system and method for nucleic acid purification. More specifically, it relates to a simple and rapid system and method for the extraction and purification of plasmid DNA from cells.

### BACKGROUND OF THE INVENTION

Plasmids are double-stranded supercoiled DNA molecules that range in size from 1 kb to more than 200 kb. Plasmids are useful tools in genetic engineering. They are widely used as vectors to carry foreign DNA; such that the foreign DNA is amplified and isolated or expressed. Plasmid DNA has also been utilized in the development of vaccines and in gene therapy.

The analysis and in vitro manipulation of plasmid DNA is typically preceded by an isolation step in order to free the nucleic acid from unwanted cellular contaminants which may interfere with subsequent processing procedures. A mini-scale sample preparation from an overnight bacterial culture of 1-5 ml generates more than enough plasmid DNA (˜few micrograms) for many of these applications.

The most common plasmid DNA extraction protocols exploit reagents originally developed by Birnboim and Doly (Birnboim, H. C. and Doly, J., Nucl. Acids Res. 7, 1513 (1979)), to separate supercoiled plasmid DNA from bacterial genomic DNA, RNA and protein. These reagents, developed many years ago, work on the principle of sequential use of three buffers, commonly referred to as buffer I, II and III. They each have distinct compositions to bring about plasmid enrichment and separation from contaminants. Buffer I is used to resuspend the bacterial pellet obtained by an initial centrifugation step of an appropriate bacterial culture. Once resuspended, buffer II is added which contains SDS detergent and NaOH. These components lyse the bacteria and denature the genomic DNA (pH>12). Buffer III typically contains a chaotrope to further denature protein, the chaotrope also promotes binding of plasmid DNA to the silica matrix commonly used in spin columns. Buffer III also usually contains potassium acetate to rapidly neutralize the combined solutions. The addition of buffer III causes contaminants to “crash-out” of solution owing to the formation of insoluble complexes driven by rapid re-naturation of genomic DNA and the potassium salt of the detergent.

The insoluble flocculant material has traditionally been removed by a centrifugation step to “pack” the flocculant material at the bottom and side of a centrifugation tube (See, e.g. ILLUSTRA™ plasmidPrep Mini Spin Kit, GE Healthcare, Piscataway, N.J.). The clarified plasmid-containing solution is subjected to a chromatographic separation. For mini-scale purification, the clarified solution is usually applied to a minispin column containing a glass fiber matrix or silica membrane. Plasmid DNA binds to the column in the presence of a chaotrope, while soluble impurities do not bind. After the soluble impurities are washed off, the plasmid DNA are eluted with an appropriate elution buffer.

Recently, alternative buffer compositions have been developed for plasmid DNA extractions based upon 96-well plates, which minimizes the formation of flocculants during bacterial lysis (DIRECTPREP™ 96 Miniprep Kit, Qiagen Inc., Valencia, Calif.). The use of these buffers generates little precipitated cellular components and eliminates the need to remove the flocculants before column loading of the DIRECTPREP™ 96-well plates. A 96-well plate pre-filter is used to capture any residual precipitants from clogging the silica membrane. However, the columns may become clogged if the cell density is high.

It is advantageous to further simplify the process therefore to provide a more efficient plasmid DNA purification method.

### SUMMARY OF THE INVENTION

In general, the instant invention provides improved methods, systems and kits for rapid isolation of plasmid DNA from plasmid containing cells.

In one aspect, the invention features a method for the rapid isolation of plasmid DNA, including: a) collecting plasmid-containing cells and resuspending them in an aqueous buffer; b) incubating the resultant mixture with a lysis/denaturation solution to lyse the cells and denature DNA; c) neutralizing the mixture with a renaturation solution to generate a renatured mixture of dissolved plasmid DNA and flocculants containing insoluble genomic DNA and cellular debris; d) loading the renatured mixture directly to a modified spin column without first removing the flocculants from the mixture, which column having a pre-filtration disc (e.g., pre-filter) on top of a matrix; e) passing loaded sample mixture through the column such that the flocculants are packed on top of the pre-filtration disc while plasmid DNA binds to column matrix; f) washing the column with a wash solution to remove soluble impurities; and g) eluting plasmid DNA from the column with an elution buffer. It has been found surprisingly that by introducing an integral pre-filtration disc, there is no longer a need to remove flocculants containing cellular debris prior to loading the spin column. Instead, the flocculants stay on top of the pre-filtration disc throughout the purification process and do not interfere with subsequent wash or elution of the plasmid DNA.

In a second aspect, the invention provides a modified spin column for the rapid isolation of plasmid DNA from plasmid-containing cells, comprising: a matrix; a support filter underneath the matrix; a pre-filter on top of the matrix; and a housing for the matrix and filters. Preferably, the modified spin column contains a glass fiber matrix and the pre-filter and support filters are made of porous sintered polyethylene. In a variation of the modified spin column, a depth filter is included between the separation matrix and the pre-filter to further enhance the performance of the system.

In another aspect, the invention provides kits for rapidly isolating plasmid DNA, including the modified spin column, reagents and user manual.

Certain aspects of the invention allow simultaneous isolation of a large number of different plasmids. The modified spin columns can be joined together to take the form of a microtiter plate. By this kind of an arrangement, a number of different plasmid containing cell cultures can be processed simultaneously. It is noted that all centrifugation steps can be replaced with vacuum.

The above and further features and advantages of the instant invention will become clearer from the following detailed description and claims.

### BRIEF DESCRIPTION OF THE DRAWINGS

FIG. 1 shows a schematic diagram of the modified spin column according to one embodiment of the invention.

FIG. 2 shows a gel image of four samples prepared according to one example of the invention, before (left) and after (right) a HindII digest. 400 ng of DNA was used for each digest, with 1 unit of HindII, and incubated at 37° C. for 2 hours. Far left: markers.

FIG. 3 shows a schematic diagram of the modified spin column according to a variation of one embodiments of the invention. A depth filter is included between the pre-filter and the main separation matrix.

FIG. 4 shows a gel image of plasmid DNA isolated using the modified combination pre-filter/depth filter systems according to the scheme of FIG. 3. The numbers represent either controls or a particular combination according to Table 1.

### DETAILED DESCRIPTION OF THE INVENTION

The invention features improved processes, systems and kits for rapidly isolating plasmid DNA from plasmid containing cells, in particular for downstream applications in molecular biological research, such as cloning and sequencing. As used herein, the term “plasmid” refers to supercoiled DNA molecules (single or double stranded) that are maintained in a host cell separate from the host cell genome. Plasmids can be of a high copy number or low copy number and can carry any gene or external piece of DNA, either genomic or synthetic, encoding protein or peptide of interest, from any source.

In general, the improved process for isolating plasmid DNA includes modification of a spin column such that it eliminates the need to remove the insoluble flocculant cellular debris generated from the lysis of the cells, before loading the column, this simplifies the work flow and shortens the protocol significantly. A spin column for plasmid DNA isolation generally contains a separation matrix placed on and supported physically by a disc of porous material more commonly referred to as a frit, typically made of sintered polyethylene. The holes so formed during the production of the frit material allow the unhindered passage of aqueous solutions and more importantly aqueous solutions containing plasmid DNA. The frit material is inert and does not interact to any great extent with DNA. The separation matrix is preferably a glass fiber matrix or a silica membrane. Alternatively, the matrix is a zeolite, or an organic matrix such as a resin or polymer.

One embodiment of the invention includes a modification of the spin column with the addition of an integral pre-filter on top of the separation matrix. One example of a pre-filter is a porous sintered polyethylene or polypropylene, similar to the support frit underneath the separation matrix.

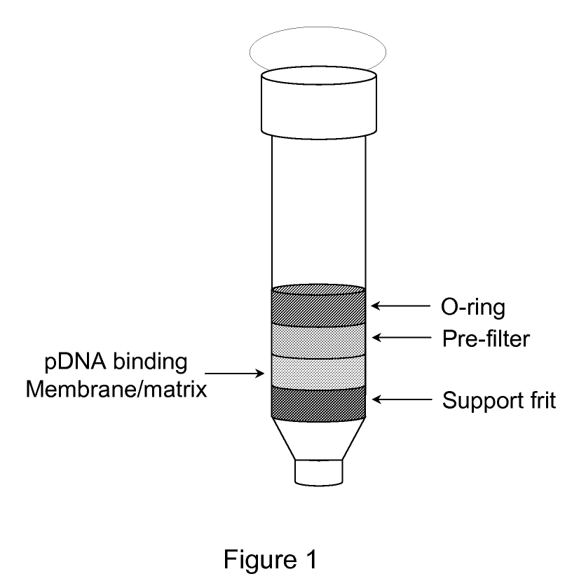

The use of the pre-filtration disc does not have to be the same composition as the lower supporting frit and indeed an optimal column might be composed of alternative materials having different filtration/binding characteristics. A thinner “pre-filter” material (e.g., cellulose absorbent paper or polypropylene mesh) may allow improved assembly of the column where sheets of appropriate components are layered together prior to die-cutting and positioning within a column moulding. Optionally, an 0-ring can be used to secure the “pre-filter” (FIG. 1).

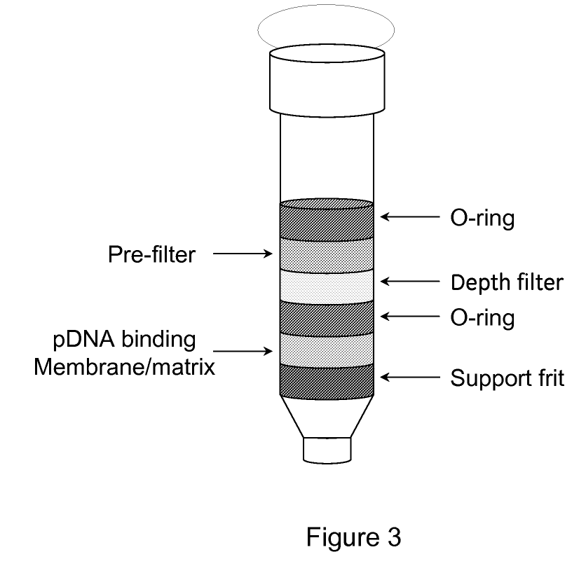

A variation of the embodiment additionally includes a depth filter between the pre-filter and the separation matrix (FIG. 3). A combination of both a pre-filter and a depth filter further reduces residual contaminant flow-through from the pre-filter, thus is preferable for certain applications. A preferred depth filter is one that captures any residual flow-through contaminants from the pre-filter yet does not retain plasmid DNA during elution. A suitable depth filter is a glass microfiber filter.

Plasmids isolated in accordance with the invention can be of any origin. Most commonly, microorganisms like bacteria, such as Escherichia coli (E. coli), are used for culturing the plasmids, but the use of host cells is not limited and can be prokaryotic or eukaryotic cells. The host cells harboring the plasmid can be cultivated in a number of ways well known in the art, e.g. in incubator, bioreactor, fermentor etc. The plasmid isolated according to the invention can be of virtually any size, e.g. in the range of about 1 kb up to about 20 kb. As an upper limit, the isolation of cosmids and artificial chromosomes is also encompassed, the size of which may be up to about 50 kb and 500 kb, respectively.

The modified spin column is suitable for extraction of plasmid DNA from standard cultures of bacteria. The inclusion of a pre-filter within a spin column eliminates the need to remove flocculant material generated by alkaline lysis, prior to addition of sample to the DNA-binding column. This modified column is especially suited for the so called miniprep of plasmid DNA from 1-5 ml overnight culture. For a miniprep, traditionally, lysate is clarified by a 5-10 minute spin in a micro-centrifuge before addition of the clarified lysate to the microspin column. Using the modified device, two steps are removed from the process without affecting quality of isolated product. Purification of plasmid DNA with the modified device can now be done in 6-8 minutes for a miniprep, compared to traditional process which typically takes about 20 minutes to complete.

The modified spin column is also suited for the preparation of plasmid DNA in a larger scale. For example, between 10-50 ml overnight culture could be used as a starting material, and larger spin columns are devised to accommodate the increased volume of the lysate. A modified, larger column with an integral prefilter achieves similar benefits as a modified microspin column.

During the experimentation it was found that the use of the pre-filter modified microspin column in combination with a fixed-angle microcentrifuge enabled the insoluble flocculent material to be pelleted “over to one side” so that occlusion of the frit pre-filter was less likely to occur. Even without a fixed-angle rotor, using a vacuum that distributes flocculant material across the entire surface of the frit, good quality plasmid DNA is still obtained that can be digested and sequenced.

By deploying the modified spin column, it has been possible to achieve multiple benefits. First, it enables the addition of lysed sample to the modified column without removal of flocculants (pre-processing). It also ensures total utilization of sample without incurring transfer losses owing to pre-processing. It further provides an improvement in the ease of use and time for completion, speeding up the process by more than 50%. The introduction of a pre-filter also stabilizes the separation matrix, known to be fragile and liable to partial fragmentation.

Methods for isolating plasmid DNA generally starts from culturing the host cells containing the plasmid. When the culture is ready, the cells are recovered by e.g. centrifugation or filtration. The cells can be stored, for example in a freezer, or processed immediately. The process for isolating plasmid DNA includes first collecting plasmid-containing cells and resuspending them in an aqueous buffer; then incubating with a lysis/denaturation solution to lyse the cells and denature DNA; followed by neutralizing the mixture with a renaturation solution to generate a renatured mixture of dissolved plasmid DNA and flocculants containing insoluble genomic DNA and cellular debris. In one aspect, the improved method includes loading the renatured solution with the flocculants directly to a modified spin column having an integral pre-filtration disc (pre-filter) on top of the separation matrix. The solution is then passed through the modified spin column by centrifugation or vacuum, such that the flocculants are packed on top of the pre-filtration disc while plasmid DNA binds to separation matrix. The modified spin column is washed with a wash solution to remove soluble impurities; and plasmid DNA is eluted from the column with an elution buffer. It is surprisingly discovered that although the flocculants remain packed on top of the pre-filter during the washing and elution steps, high quality plasmid DNA is isolated that is suitable for subsequent molecular biology analysis.

The protocols for cell lysis and denaturation of cellular debris are well known. A particularly useful aqueous buffer for resuspending plasmid-containing cells contains an isotonic buffer (e.g. a Tris buffer; or a sucrose or glucose solution), a chelating agent (e.g. ethylenediaminetetraacetic acid (EDTA) or (CDTA)) and an RNAse. This buffer may also optionally include lysozyme to further weaken cell walls. After the cells are resuspended, the cells are lysed and linear DNA is denatured, preferably by incubation in an alkaline lysis solution. Thorough lysis and denaturation can be accomplished by mixing the resuspended cells with a sodium hydroxide, sodium dodecyl sulfate solution. A third, renaturation solution (e.g. an acetate buffered solution, containing a chaotropic salt) is then added to yield a mixture containing plasmid DNA, insoluble clots of linear DNA and cellular debris.

According to one aspect of the invention, the renatured mixture of dissolved plasmid DNA and insoluble flocculants are loaded to the modified spin column. Through vacuum or centrifugation, liquids in the mixture passes through the column, leaving on top of the pre-filter a packed layer of flocculants, in the meantime plasmid DNA binds to column matrix. A wash solution is then applied to remove soluble impurities; and plasmid DNA is then eluted from the modified spin column with an elution buffer. The flocculants remain packed on top of the pre-filtration disc during the washing and eluting steps but does not affect the quality of the plasmid DNA isolated.

The addition of a depth filter between the pre-filter and the separation matrix results in slightly better quality DNA. Thus it is preferable to include a depth filter in the modified spin column for certain preparations. The workflow, however, does not change from the protocol which includes the pre-filter only.

Certain aspects of the invention allow simultaneous isolation of a large number of different plasmids. The modified spin columns can be joined together to take the form of a microtiter plate. Especially preferred are microtiter plates in the 96 well format. By this kind of an arrangement, a large number of plasmid containing cultures can be processed simultaneously. It is noted that all centrifugation steps can be replaced with vacuum.

### EXAMPLES

The following examples serve to illustrate the plasmid DNA purification processes according to embodiments of the present invention and are not intended to be limiting.

### 1. The Protocol

The protocol is suitable for the rapid extraction and purification of plasmid DNA from 1.5 ml cultures of E. coli. The procedure can be completed in less than 10 minutes to yield plasmid DNA with a purity and quality compatible with many common molecular biology techniques, including cloning, restriction enzyme digestion, PCR amplification and DNA sequencing.

The plasmid DNA yield from a freshly grown E. coli strain containing a high copy number plasmid (>300 copies/cell) and grown to A₆₀₀ approximately 2.5 is typically 4 to 6 μg (A₂₆₀/A₂₈₀>1.8).

The protocol utilizes a simple plasmid DNA purification process, employing a modified alkaline cell lysis procedure and a silica-based membrane. No organic solvents are used; instead, chaotropic salts are included to denature protein components and promote the selective binding of plasmid DNA to the silica membrane. Denatured insoluble contaminants are retained on top of pre-filter, while soluble contaminants are easily removed by subsequent washing. The purified plasmid DNA is eluted in a low ionic strength buffer, at a plasmid concentration suitable for most molecular biological applications.

The following provides a step by step protocol:
1. Transfer 1.5 ml from a fresh overnight culture to a microcentrifuge tube. To pellet bacteria, centrifuge (13 000×g) for 30 seconds. Discard supernatant and re-centrifuge. Remove any residual supernatant using a pipette.
2. Thoroughly resuspend the pellet by adding 150 μl lysis buffer (100 mM Tris-HCl pH7.5; 10 mM EDTA; 0.2 mg/ml RNase A), and either vortexing, pipetting up and down or scraping the base of the microcentrifuge tube across the surface of an empty pipette tip rack.
3. Cell lysis—Add 150 μl lysis buffer (200 mM NaOH; 1% SDS) and mix immediately by gentle inversion (approximately 5 times) until solution becomes clear and viscous.
4. Neutralisation—Add 300 μl neutralization buffer (4.4M Guanidine HCl, 0.65M potassium Acetate and 3.1 M Glacial Acetic Acid), and mix immediately by gentle inversion until the precipitate is evenly dispersed.
5. Transfer the neutralized mixture to the modified microspin column (approximately 600 μl). Close the lid of the column gently. Centrifuge (13 000×g) for 30 seconds. Discard the flow through by emptying the collection tube.
6. Wash the column with 600 μl wash buffer (2 mM Tris-HCl pH8; 0.2 mM EDTA and 80% ethanol) and centrifuge (13 000×g) for 30 seconds. Discard the flow-through and repeat the wash one more time with a 60 second spin.
7. Move the modified microspin column into a fresh microcentrifuge tube and add 100 μl elution buffer (10 mM Tris-HCl pH8) directly onto the centre of the column. Incubate the column for 30 seconds at room temperature. Microcentrifuge (2 000×g) for 60 seconds to recover the plasmid DNA as flow through in the microcentrifuge tube.

Purified plasmid DNA concentration should be determined by UV spectrophotometry (A₂₆₀) and through comparison with a known standard by agarose gel electrophoresis and subsequent densitometric analysis. If available, the UV spectrophotometric ratios A₂₆₀:A₂₈₀ and A₂₆₀:A₂₃₀ provide a limited indication of purity as measures of protein and salt contamination.

### 2. Purification of Plasmid DNA Using a Modified Microspin Column Containing a Prefilter

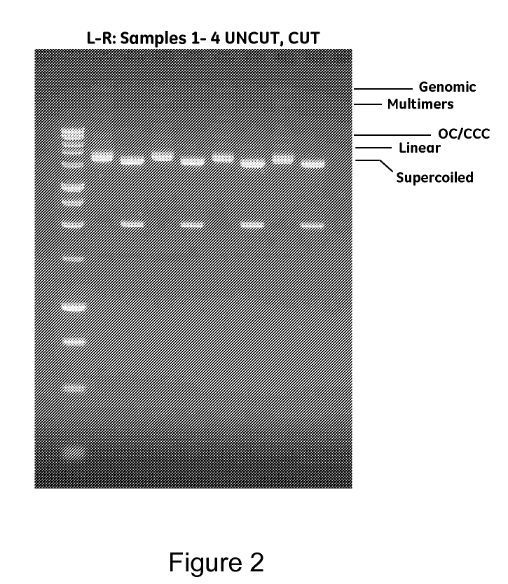

Overnight cultures of E. coli TOP10 transformed with pCORON1002-EGFP-C1 were processed following the protocol described above. Four individual cultures were prepared and plasmid DNA was isolated according to the protocol. Modified microspin columns contained a pre-filtration disc of a porous, sintered polyethylene. The samples had a mean yield of 6.7 μg. The plasmids are suitable for downstream molecular biology applications as illustrated by restriction enzyme digestion (FIG. 2).

### 3. Purification of Plasmid DNA Using a Variation of the Modified Microspin Column

To further reduce extraction time whilst maintaining the purity/quality of the isolated DNA to a level comparable to that generated using traditional microspin systems, the inclusion of a depth column between the pre-filter and the main separation matrix was tested (FIG. 3).

The above protocol was used for plasmid DNA isolation, with slight modification. Briefly, 125 μl re-suspension buffer and lysis buffer, respectively, was used for each culture, while 250 μl neutralization buffer was used. Crude lysate was added directly onto the integral filtration/plasmid DNA binding column and centrifuged at 13,000 g for 60 s in a microcentrifuge. The columns were washed twice with 400 μl wash buffer before DNA elution. Absorbance data was determined using a Nanodrop ND1000 spectrophotometer.

A number of pre-filter and depth filter combinations were tested, using a silica membrane column as the main plasmid DNA binding matrix (the column from ILLUSTRA™ plasmidPrep Mini Spin kit). To compare the quality and yield with traditional protocols, controls were included. The control experiments were performed following manufacturer's protocols, except the pre-filter only control which was performed following the current protocol. The depth filter used was the Whatman GF/B glass microfibre depth filter. Table 1 lists the pre-filters tested in combination with the Whatman GF/B glass microfibre depth filter, and the control experiments performed.

TABLE 1
Summary of pre-filter/depth filter combinations tested.

| | Control or Pre-filter | Depth filter |
|---|---|---|
| 1 | Control - Qiagen QIAPREP ™ Spin | no |
| | Mini kit (Cat. 27106) | |
| 2 | Control - ILLUSTRA ™ plasmidPrep | no |
| | Mini Spin kit (Cat. 28-9042-69) | |
| 3 | Control - ILLUSTRA ™ plasmidPrep | no |
| | Mini Spin kit with Macherey Nagel QUICKPURE ™ | |
| | column (Cat 406184) PE frit as pre-filter only | |
| 4 | Macherey Nagel QUICKPURE ™ column | yes |
| | PE frit (Cat 406184) | |
| 5 | Pall Corp. LAS (40 um) (P/N 0270550201) | yes |
| 6 | Pall Corp. Absorbent Paper 165 (P/N S70009) | yes |
| 7 | Handee Spin column PE frit (25 um) (P/N 89868) | yes |
| 8 | Porex X-4920 PE frit (10-20 um pore size) | yes |
| 9 | Macherey Nagel 96-well mesh | yes |

For each pre-filter/depth filter combination (or control experiment), at least three parallel experiments were run. It was found that with an integral pre-filter/depth filter combination, the time needed to complete a plasmid DNA isolation experiment was about 7.5 min. In comparison, the ILLUSTRA™ plasmidPrep Mini Spin kit took about 9 min to complete, while the QIAPREP™ Spin Mini kit took about 19 min to complete. In general, the modified system with both a pre-filter and a depth filter generated comparable amount of plasmid DNA as the control extractions irrespective of the material used as the pre-filter.

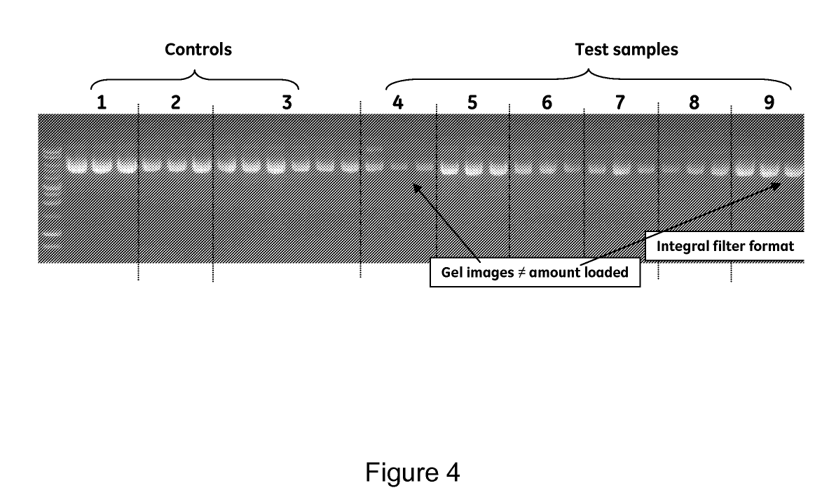

The quality of the isolated plasmids were also comparable to the ones isolated using the control kits. Low level of protein contamination was observed. The amount of particulates in the final elution was also comparable to control extractions. Salt levels were lower than the control QIAPREP™ or ILLUSTRA™ plasmidPrep kit. The majority of native plasmid DNA was in the supercoiled configuration (FIG. 4; 300 ng of DNA loaded on 1% agarose gel)). Therefore in general the quality of the isolated plasmid DNA was comparable to control extractions.

The modified microspin column with both a pre-filter and a depth filter combines the speed of a pre-filter only system (i.e. 7.5 min) with quality associated with traditional spin extraction methods/kits. Even though the Whatman GF/B micro-fibre depth filter probably binds some plasmid DNA in the presence of the chaotrope, in an integral filter format the plasmid DNA can be recovered during the final elution step.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. A method for the rapid isolation of plasmid DNA from plasmid-containing cells, including:
a) collecting said plasmid-containing cells and resuspending them in an aqueous buffer;
b) incubating the resultant mixture with a lysis/denaturation solution to lyse the cells and denature DNA;
c) neutralizing the mixture from step b) with a renaturation solution to generate a renatured mixture of dissolved plasmid DNA and flocculants containing insoluble genomic DNA and cellular debris;
d) loading the renatured mixture from step c) directly to a modified spin column without first removing said flocculants from the mixture, which column containing a pre-filtration disc on top of a matrix and a support filter underneath the matrix;
e) passing loaded sample mixture through the modified spin column such that the flocculants are packed on top of said pre-filtration disc while plasmid DNA binds to column matrix;
f) washing said modified spin column with a wash solution to remove soluble impurities; and
g) eluting plasmid DNA from said modified spin column with an elution buffer;
wherein said flocculants remain packed on top of said pre-filtration disc during said washing and eluting steps.

2. The method of claim 1, further including desalting said eluted plasmid DNA.

3. The method of claim 1, wherein the matrix in said modified spin column is a glass fiber matrix, a silica membrane or a zeolite.

4. The method of claim 1, wherein the matrix in said modified spin column is an organic matrix.

5. The method of claim 1, wherein said pre-filtration disc is a disc of porous, sintered polyethylene or polypropolylene.

6. The method of claim 1, wherein said pre-filtration disc has the same composition as the support filter underneath the matrix.

7. The method of claim 1, wherein each of steps e) through g) is assisted by vacuum.

8. The method of claim 1, wherein each of steps e) through g) is assisted by centrifugation.

9. The method of claim 1, wherein said modified spin column further comprises a depth column below said pre-filtration disc but above said matrix.

10. The method of claim 1, wherein the aqueous buffer in step a) is comprised of an isotonic buffer, a chelating agent and an RNAse.

11. The method of claim 10, wherein the isotonic buffer is selected from the group consisting of: a Tris buffer, a sucrose solution or a glucose solution.

12. The method of claim 10, wherein the aqueous buffer additionally comprises a lysozyme.

13. The method of claim 4, wherein the matrix is a resin or polymer.

14. The method of claim 1, wherein the lysis solution is alkaline.

15. The method of claim 14, wherein the lysis solution comprises sodium hydroxide and sodium dodecyl sulfate.

16. A modified spin column for the rapid isolation of plasmid DNA from plasmid-containing cells, comprising: (a) a matrix; (b) a support filter underneath said matrix; (c) a pre-filtration disc on top of said matrix; and (d) a housing for said matrix and filters.

17. The modified spin column of claim 16, wherein the matrix is a glass fiber matrix, a silica membrane or a zeolite.

18. The modified spin column of claim 16, wherein the matrix is an organic matrix.

19. The modified spin column of 18, wherein the matrix is a resin or polymer.

20. The modified spin column of claim 16, wherein both the support filter and the pre-filtration disc are of the same composition and are made of porous sintered polyethylene.

21. The modified spin column of claim 16, further comprising a depth filter between said pre-filtration disc and said matrix.
